(19) United States
(12) Patent Application Publication
(10) Pub. No.: US 2021/0290885 A1
(43) Pub. Date: Sep. 23, 2021

(54) APPARATUS, SYSTEMS AND METHODS FOR DELIVERING CONDITIONED AIR TO A PATIENT LUNG INTUBATION TUBE

(71) Applicant: Rapid Medical Parts, Inc., Buffalo, NY (US)

(72) Inventor: James Allen Regenor, East Aurora, NY (US)

(73) Assignee: Rapid Medical Parts, Inc., Buffalo, NY (US)

(21) Appl. No.: 17/210,029

(22) Filed: Mar. 23, 2021

Related U.S. Application Data
(60) Provisional application No. 62/993,376, filed on Mar. 23, 2020.
(60) Provisional application No. 63/004,909, filed on Apr. 3, 2020.
(60) Provisional application No. 63/006,367, filed on Apr. 7, 2020.
(60) Provisional application No. 63/016,584, filed on Apr. 28, 2020.
(60) Provisional application No. 63/017,386, filed on Apr. 29, 2020.

(51) Int. Cl. A61M 16/12; A61M 16/06; A61M 16/04; A61M 16/20; A61M 16/16

(52) U.S. Cl. CPC A61M 16/12; A61M 16/06; A61M 16/0672; A61M 2016/0027; A61M 16/208; A61M 16/16; A61M 2202/0208; A61M 16/04

(57) ABSTRACT

An apparatus for supplying oxygenated conditioned air to one or more patients includes an positive airway pressure inlet air source to provide a pressurized inlet air. A pressurized oxygen source provides a pressurized oxygen gas to be mixed with the inlet air. A sensor panel including one or more sensors is communicatively coupled to each of the inlet air source and the pressurized oxygen source. The sensor panel selectively controls metering of the pressurized oxygen gas from the pressurized oxygen source so that a partial pressure of the pressurized oxygen gas within the oxygenated conditioned air is maintained within a predetermined concentration range. An oxygenated conditioned air output is coupled to a respective intubation tube to supply the oxygenated conditioned air to the one or more patients.

### FIELD OF THE INVENTION

The present invention relates to an apparatus, and methods and systems for providing breathing assistance to a patient, and more particularly relates to an apparatus, methods and systems that employ a continuous positive airway pressure (CPAP) or bi-level positive airway pressure (BiPAP) or BiPAP Spontaneous Timed (BiPAP ST) apparatus for supplying conditioned, oxygenated air to the lungs of an intubated patient or patients, and still more particularly, to an apparatus, method and system for providing breathing assistance using a positive pressure air or oxygen source without requiring a CPAP, BiPAP or BiPAP ST unit.

### BACKGROUND OF THE INVENTION

During widespread medical emergencies, such as pandemic, the need for hospital respirators may exceed the number of available respirator units. The leads to unnecessary deaths and undue hardship on hospital doctors and staff, as well as families of those lost. One approach to overcome this deficiency may be to repurpose equipment designed to treat sleep apnea. That is, there are hundreds of thousands of positive airway pressure systems continuous positive airway pressure (CPAP) or bi-level positive airway pressure (BiPAP) or BiPAP Spontaneous Timed (Bi-PAP ST) in clinics and homes that may be requisitioned in an emergency situation. However, these systems are not configured for use with intubated patients.

### SUMMARY OF THE INVENTION

The present invention provides in a first aspect an apparatus, system and method for supplying conditioned air to an intubation tube. A continuous positive airway pressure (CPAP) apparatus delivers conditioned air to a regulator/mixing valve wherein oxygen may be introduced into the conditioned air stream at the desired concentration. A sensor panel is operable to sense parameters of the air stream including, for example, pressure and oxygen concentration. The regulator and sensors may communicate so as to maintain the preset operating parameters of the air stream.

In another embodiment, rather than a CPAP, the invention utilizes a BiPAP or BiPAP ST. A BiPAP delivers two set pressures, a higher pressure for inhalation and a lower pressure for exhalation. In the Bi-PAP ST, the device triggers to IPAP (inhalation positive air pressure) on patient inspiratory effort but a “backup” rate is also set to ensure that patients still receive a minimum number of breaths per minute if they fail to breathe spontaneously.

In another embodiment, multiple patients may be individually assisted using a single CPAP/BiPAP apparatus. A manifold-and-tubing may be used to provide patient-specific conditioned air, while also minimizing any possibility of cross-contamination between patients.

In still another embodiment, an apparatus is provided that delivers pressurized air to a regulator/mixing valve wherein additional oxygen may be introduced into the air stream at the desired concentration. A sensor panel is operable to sense parameters of the air stream including, for example, pressure and oxygen concentration at multiple locations along the flow path. The regulator and sensors may communicate so as to maintain the preset operating parameters of the air stream.

In the following discussions, the term “PAP” shall collectively refer to any positive air pressure apparatus, including without limitation a CPAP, BiPAP or BiPAP ST apparatus, unless the features described in combination are only available/possible using specific apparatuses, which the applicant will attempt to distinguish, as needed.

Additional objects, advantages and novel aspects of the present invention will be set forth in part in the description which follows, and will in part become apparent to those in the practice of the invention, when considered with the attached figures.

### DESCRIPTION OF THE DRAWING FIGURES

The above-mentioned and other features and advantages of this invention, and the manner of attaining them, will become apparent and be better understood by reference to the following description of the invention in conjunction with the accompanying drawing, wherein:

FIG. 1 is a diagrammatic view of a first embodiment of the invention which utilizes a PAP apparatus with post-apparatus mixing and supplemental oxygenation;

FIG. 2 is a diagrammatic view of an alternative embodiment of the invention which utilizes a PAP apparatus with metered oxygen addition at the apparatus inlet;

FIG. 3 is a diagrammatic view of another embodiment of the invention which utilizes a PAP apparatus with post-apparatus mixing and supplemental oxygenation with an inline check valve to prevent backflow;

FIG. 4 is a diagrammatic view of an alternative embodiment of the invention shown in FIG. 3;

FIG. 4A is a diagrammatic view of another alternative embodiment of the invention shown in FIG. 3;

FIG. 4B is a diagrammatic view of still another alternative embodiment of the invention shown in FIG. 3;

FIG. 4C is a diagrammatic view of yet another alternative embodiment of the invention shown in FIG. 3;

FIG. 4D is a diagrammatic view of another embodiment of the invention which does not require a PAP apparatus to supply pressurized source gas;

FIG. 5 is a diagrammatic view of the air/oxygen interface used within the alternative embodiment shown in FIG. 4;

FIG. 6 is a representative plot of a respiration cycle;

FIG. 7 is a diagrammatic view of still another alternative embodiment of the invention which utilizes a PAP apparatus with metered oxygen and a flow splitter;

FIG. 8 is a diagrammatic view of yet another alternative embodiment of the invention which utilizes a PAP apparatus and oxygen equipped with quantity measuring valves; and

FIG. 9 is a diagrammatic view of an additional embodiment of the invention which utilizes a PAP apparatus for assisting multiple patients using a single apparatus.

### DETAILED DESCRIPTION OF A PREFERRED EMBODIMENT

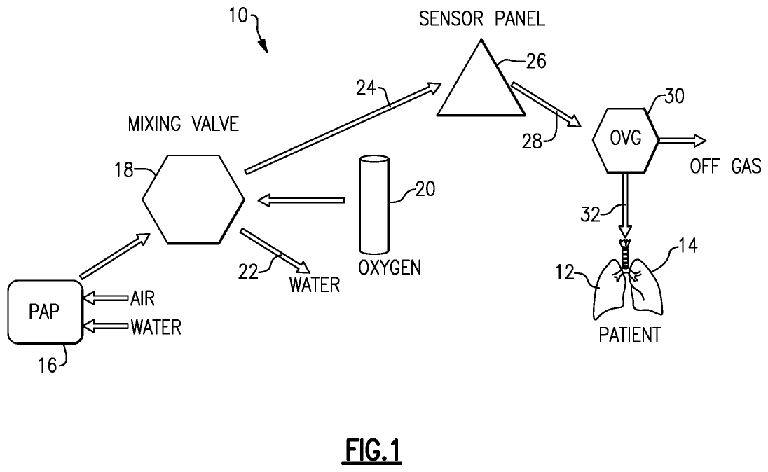

Referring to FIG. 1, there is seen a system designated generally by the reference numeral 10. A patient's lungs are indicated at 12 with an intubation tube indicated at 14. System 10 may include a continuous positive airway pressure (CPAP), bi-level positive airway pressure (BiPAP) or BiPAP Spontaneous Timed (Bi-PAP ST) (collectively, “PAP”) apparatus at 16. PAP therapy is a common treatment for obstructive sleep apnea and includes a hose and mask or nosepiece to deliver constant and steady (CPAP) or bi-level (BiPAP/BiPAP ST) air pressure to the individual. In the present invention, the PAP is not connected directly to the patient, but rather is connected to a mixer valve 18 that introduces a source of oxygen 20 to the output of the PAP. The PAP may have a positive end-expiratory pressure (PEEP) of between about 5-20 cm H₂O, which is the pressure in the lungs (alveolar pressure) above atmospheric pressure (the pressure outside of the body) that exists at the end of expiration. Ambient air and water (e.g., via a reservoir tank, not shown) are supplied to the PAP 16 which outputs warm, humidified air under pressure to regulator/mixing valve 18.

The air stream, which has been conditioned by the PAP, is delivered from the PAP to the regulator/mixing valve 18 through appropriate tubing. Valve 18 includes functionality such as oxygenation of the air received from PAP 16. An oxygen source, such as indicated at 20, introduces oxygen to the PAP conditioned air stream to increase the oxygen concentration thereof to the desired level. By way of example and without limitation thereto, oxygen source 20 may include a pressurized oxygen tank or pressurized oxygen gas from a hospital supply line. Valve 18 may include an excess moisture drain 22 should the water content in the air stream exceed the desired air humidification setting.

The regulated, warm, humidified and oxygenated air stream 24 exits valve 18 and is delivered to a sensor unit 26. Unit 26 may be operable to sense one or more characteristics of air stream 24, including but not limited to the pressure, water content (humidity) and/or partial pressure of oxygen within the air stream 24. Should any sensor reading be outside of the set operating parameters of the unit 26, an alarm may sound to alert an attendant of the need for remedial action. The regulated air stream exits unit 26 and is delivered as a conditioned air stream 28 to an off-gas valve 30. Off-gas valve 30 is operable to dump excess gas to ambient via an off-gas port. The resultant conditioned air stream 32 is delivered to the patient 12 via intubation tube 14.

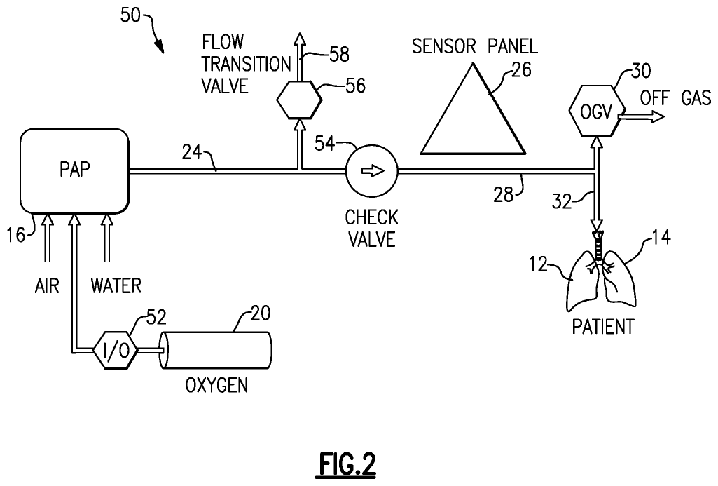

With reference to FIG. 2, another embodiment of a PAP system is designated generally by the reference numeral 50. Similar to system 10 described above, system 50 includes a PAP apparatus 16 configured to supply pressurized air to a patient's lungs 12 via intubation tube 14. Again, PAP 16 is not connected directly to the patient. However, unlike system 10 above, system 50 introduces a source of oxygen 20 to the input of PAP 16 along with ambient air and water (e.g., via a reservoir tank, not shown) so as to output regulated, warm, humidified air 24 under pressure to sensor unit 26. Supply of oxygen may be regulated via switching valve 52. A check valve 54 is place inline between PAP 16 output and sensor panel 26 to prevent backflow of any air from downstream components to PAP 16. A flow transition valve 56 may also be placed upstream of check valve 54 to dump excess flow 58 from PAP 16 to ambient.

As described above with system 10, sensor unit 26 may be operable to sense one or more characteristics of air stream 24, including but not limited to the pressure, water content (humidity) and/or partial pressure of oxygen within the air stream 24. Should any sensor reading be outside of the set operating parameters of sensor unit 26, an alarm may sound to alert an attendant of the need for remedial action. The regulated air stream exits unit 26 and is delivered as a conditioned air stream 28 to an off-gas valve 30. Off-gas valve 30 is operable to dump excess gas to ambient via an off-gas port. The resultant conditioned air stream 32 is delivered to the patient 12 via intubation tube 14.

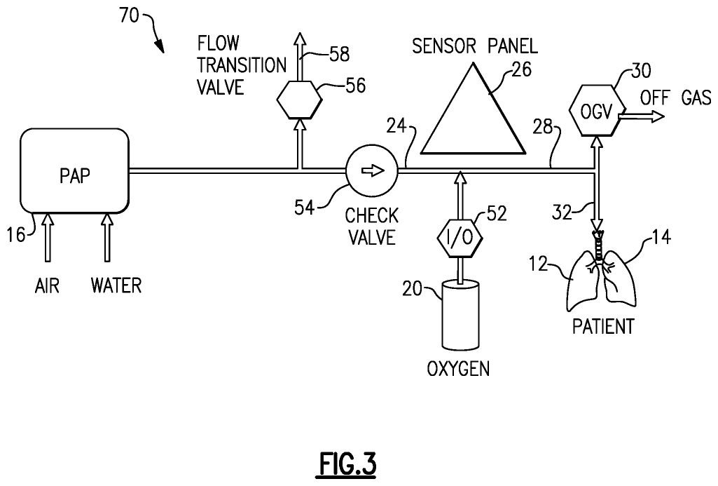

Turning now to FIG. 3, an alternative embodiment of a PAP system is designated generally by the reference numeral 70. PAP system 70 is identical to system 50 described with respect to FIG. 2, with the exception of the input location of oxygen from oxygen source 20. Rather than feed oxygen directly into PAP 16, PAP system 70 places oxygen source 20 (and its associated switching valve 52) downstream of check valve 54. Thus, after any excess flow 58 is dumped through flow transition valve 56, the air travels through check valve 54 to oxygen source 20 and sensor panel 26.

Again, sensor unit 26 may be operable to sense one or more characteristics of air stream 24, including but not limited to the pressure, water content (humidity) and/or partial pressure of oxygen within the air stream 24. In one aspect of the present invention, sensor 26 may have operational control over switching valve 52 so as to controllably inject oxygen into the air stream during an inhalation phase. However, should any sensor reading be outside of the set operating parameters of sensor unit 26, an alarm may sound to alert an attendant of the need for remedial action. The regulated air stream exits unit 26 and is delivered as a conditioned air stream 28 to an off-gas valve 30. Off-gas valve 30 is operable to dump excess gas to ambient via an off-gas port. The resultant conditioned air stream 32 is delivered to the patient 12 via intubation tube 14.

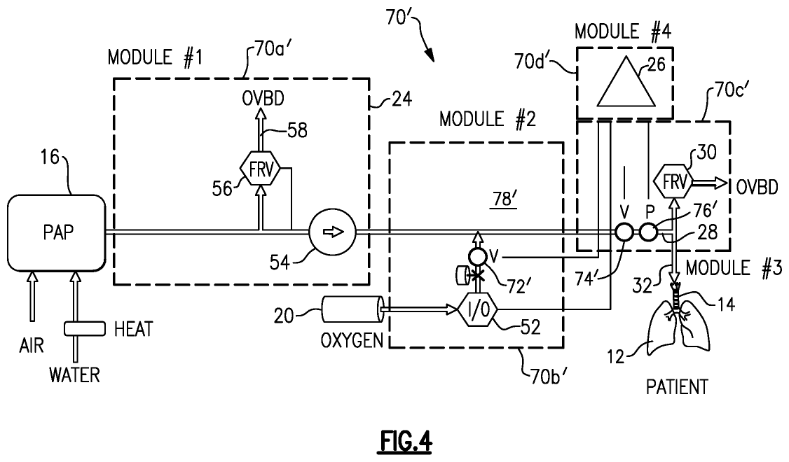

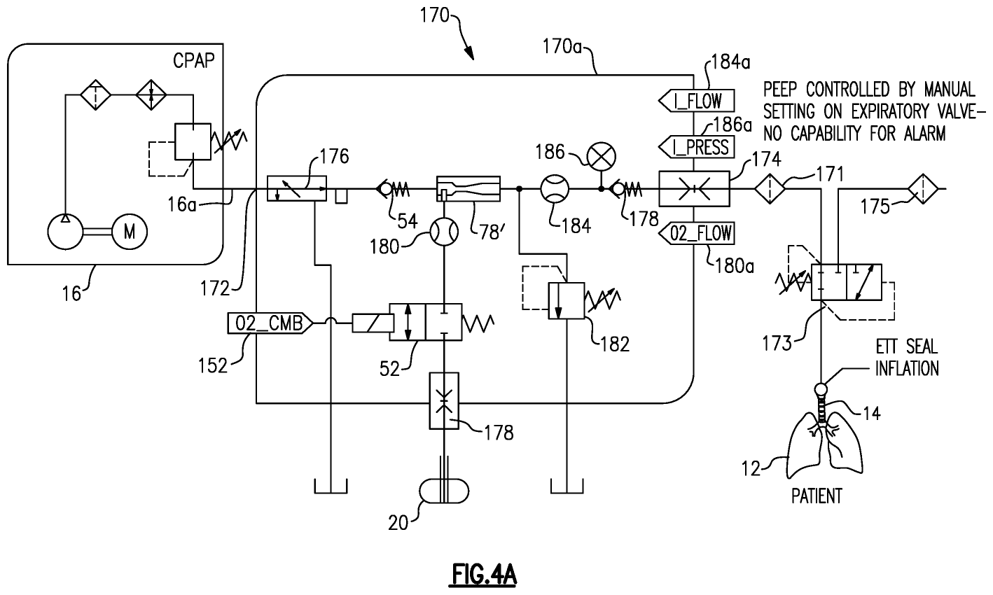

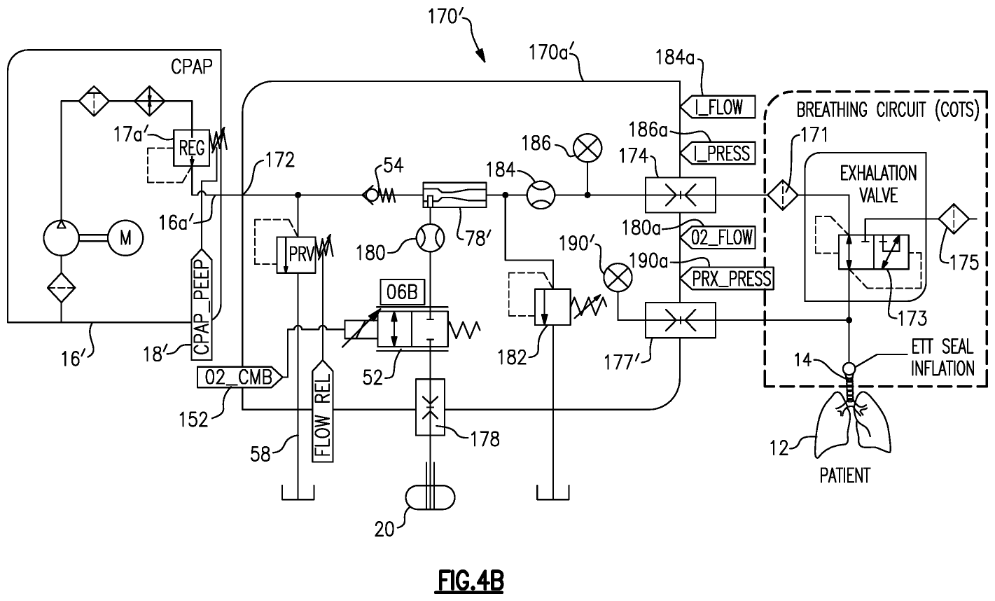

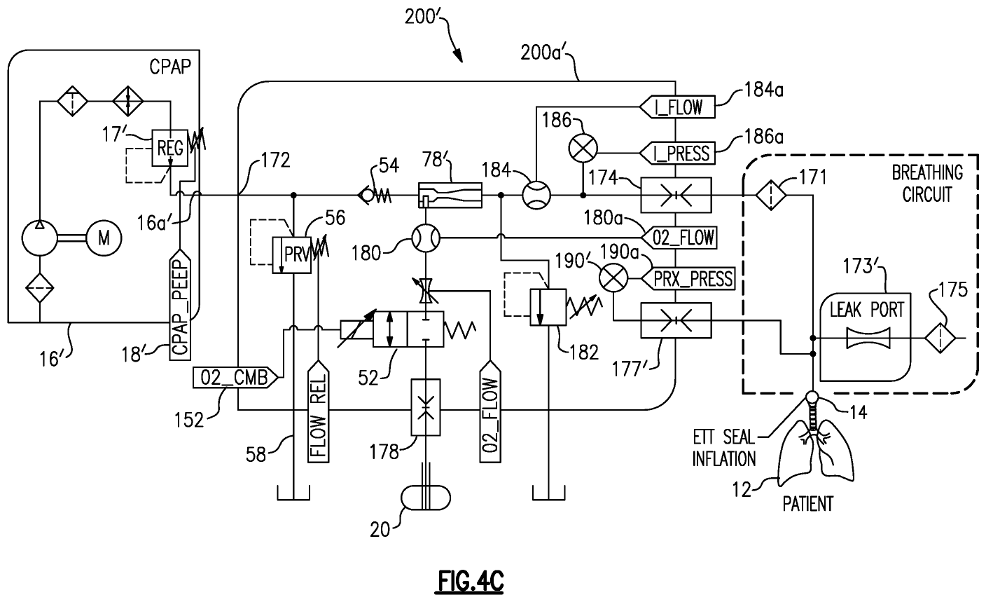

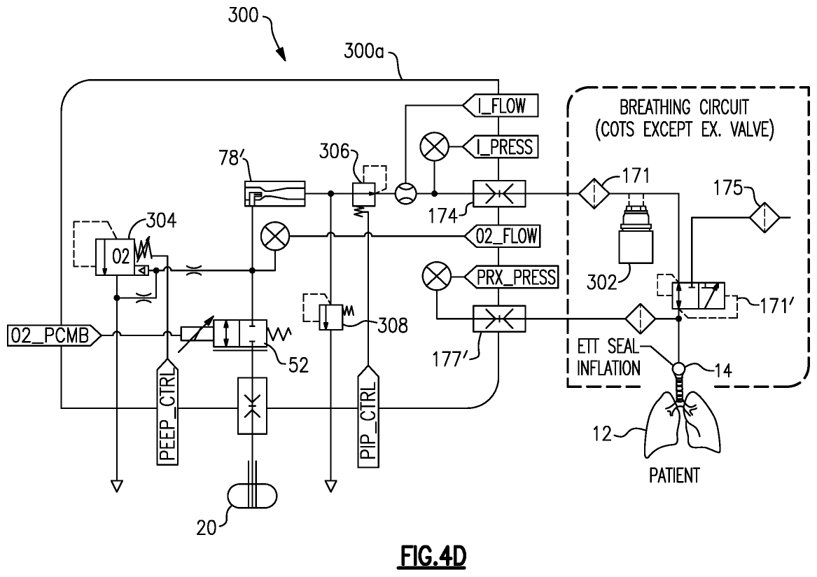

FIG. 4 shows an alternative embodiment 70′ that is substantially identical to PAP system 70 shown in FIG. 3, with system 70′ configured as a series of modules 70a′, 70b′, 70c′, 70d′. Module 70a′ comprises check valve 54 and flow transition valve 56. Module 70d′ comprises sensor panel 26. Module 70b′ comprises switching valve 52 and further includes an oxygen volume gauge 72′. Both switching valve 52 and oxygen volume gauge 72′ may be communicatively coupled to sensor panel 26. Switching valve 52 may further be operably coupled to sensor panel 26 whereby sensor panel 26 may transmit control signals to switching valve 52 to selectively open and close switching valve 52 and input pressurized oxygen from oxygen source 20 into the air stream 24. Module 70c′ comprises off-gas valve 30 and may further include volume gauge 74′ and pressure gauge 76′, each communicatively coupled to sensor panel 26.

In accordance with an aspect of the present invention, the modular design of system 70′ limits patient contact with system components to module 70c′. The remaining modules 70a′, 70b′ and 70d′ may be wiped clean between patients while a sanitized module 70c′ is introduced for each new patient. Used modules 70c′ may be sterilized between uses without requiring downtime for the remainder of the system. In this manner, system efficiency may be maximized while minimizing cross-contamination of system components.

FIG. 4A shows another alternative embodiment 170 that is substantially identical to PAP systems 70 and 70′ shown in FIGS. 3 and 4. Like system 70′, system 170 is configured as a modular system designed as a plug-and-play system. That is, respirator module 170a is configured with an inlet coupling 172 adapted to releasably couple with the outlet 16a of PAP 16. PAP 16 may be an included component of system 170 or may be provided by a home user, nursing home, medical facility and the like. Respirator module 170a may further include an outlet coupling 174 adapted to releasably couple with intubation tube 14 and patient 12. Patient 12 may be within a hospital such that intubation tube 14 and other materials (inlet filter 171, expiratory valve 173 and output filter 175) may be provided by the hospital at the time of intubation of patient 12.

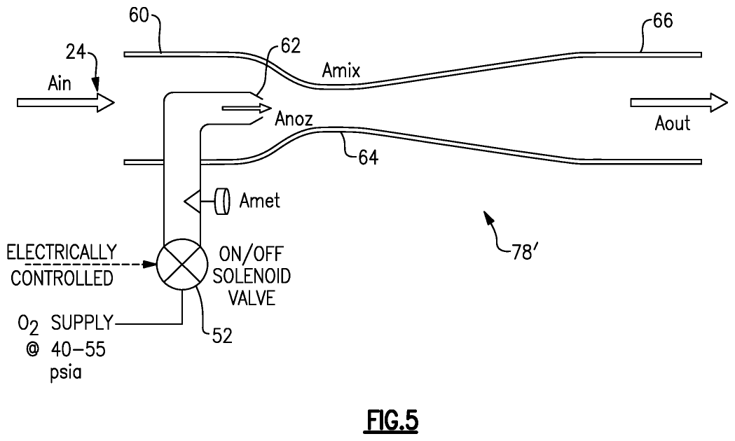

As shown in FIG. 4A, respirator module 170a may comprise a variable flow limiter 176 located between inlet coupling 172 and check valve 54. Oxygen source 20 may be coupled to respirator module 170a via coupling 178. As discussed above, oxygen source may be, for example, an external oxygen tank or from a pressurized oxygen line at a hospital. Switching valve 52 is communicatively coupled to sensor panel (e.g., sensor panel 26 as seen in FIG. 4) and configured to receive control signals 152 to open and close so as to selectively supply oxygen to patient 12, as needed. Flow meter 180 monitors output of switching valve 52 prior to reaching air/oxygen interface 78′, described in more detail below with regard to FIG. 5.

The air/oxygen mixture comprising conditioned air stream 28 may then be pressure-adjusted via variable pressure relief valve 182 before passing through flow meter 184 and pressure transducer 186. Oxygen flow 180a, air/oxygen flow 184a and air/oxygen pressure 186a may be monitored by flow meter 180, mixed air flow meter 184 and pressure transducer 186, each of which is in communication with sensor panel 26, as described above. Conditioned air stream 28 then exits respirator module 170a via outlet coupling 174 for delivery to patient 12 after passing through check valve 188.

FIG. 4B shows still another alternative embodiment 170′ that is substantially identical to PAP system 170 shown in FIG. 4A. Again, system 170′ is configured as a modular system designed as a plug-and-play system. That is, respirator module 170a′ is configured with an inlet coupling 172 adapted to releasably couple with the outlet 16a′ of PAP 16′. PAP 16′ may be an included component of system 170 or may be provided by a home user, nursing home, medical facility and the like. Additionally, PAP 16′ may be equipped with an adjustable pressure regulating valve 17′. Valve 17′ may be selectively adjustable, such as through a actuatable knob, dial, switch or slide 18′, to adjust the patient PEEP.

Respirator module 170a′ may further include an outlet coupling 174 and expiratory coupling 177′, each adapted to releasably couple with intubation tube 14 and patient 12. Patient 12 may be within a hospital such that intubation tube 14 and other materials (inlet filter 171, expiratory valve 173 and output filter 175) may be provided by the hospital at the time of intubation of patient 12.

As shown in FIG. 4B, respirator module 170a′ may comprise a flow transition valve or pressure relief valve 56 located between inlet coupling 172 and check valve 54. Valve 56 may be selectively controlled, such as via manual or electronic actuation, to adjust the output threshold of excess flow 58. Similar to respiratory module 170a, module 170a′ is coupled to oxygen source via coupling 178. As discussed above, oxygen source may be, for example, an external oxygen tank or from a pressurized oxygen line at a hospital. Switching valve 52 is communicatively coupled to sensor panel (e.g., sensor panel 26 as seen in FIG. 4) and configured to receive control signals 152 to open and close so as to selectively supply oxygen to patient 12, as needed. In accordance with one aspect of the present invention, switching valve may comprise an electronically actuatable proportional solenoid metering valve. Flow meter 180 monitors output of switching valve 52 prior to reaching air/oxygen interface 78′, described in more detail below with regard to FIG. 5.

The air/oxygen mixture comprising conditioned air stream 28 may then be pressure-adjusted via variable pressure relief valve 182 before passing through flow meter 184 and pressure transducer 186. Oxygen flow 180a, air/oxygen flow 184a and air/oxygen pressure 186a may be monitored by flow meter 180, mixed air flow meter 184 and pressure transducer 186, each of which is in communication with sensor panel 26, as described above. Conditioned air stream 28 then exits respirator module 170a via outlet coupling 174 for delivery to patient 12. Expiratory coupling 177′ fluidly couples the expiratory tube of intubation tube 14 with a pressure transducer 190′ which is in communication 190a′ with sensor panel 26 to monitor patient expiratory pressure with regard to the PAP set PEEP (pressure regulating valve 17′).

FIG. 4C shows yet another alternative embodiment 200′ that is substantially identical to PAP system 170′ shown in FIG. 4B. Again, system 200′ is configured as a modular system designed as a plug-and-play system and includes PAP 16′. Respirator module 200a′ is configured identically to respirator module 170a′ but includes an additional switching valve 52 immediately downstream of electronically actuatable proportional solenoid metering valve 52.

Respirator module 170a′ may further include an outlet coupling 174 and expiratory coupling 177′, each adapted to releasably couple with intubation tube 14 and patient 12 as described above. Patient 12 may be within a hospital such that intubation tube 14 and other materials (inlet filter 171, leak port 173′ and output filter 175) may be provided by the hospital at the time of intubation of patient 12.

FIG. 4D shows still another alternative embodiment of a pressurized air delivery system 300 that is similar to PAP systems 170, 170′, 200′ shown in FIGS. 4A-4C but does not require a PAP 16, 16′. Rather, the only pressurized source gas provided to respirator module 300a comes from oxygen source 20. Like above, system 300 is configured as a modular system designed as a plug-and-play system and includes respirator module 300a having an outlet coupling 174 and expiratory coupling 177′, each adapted to releasably couple with intubation tube 14 and patient 12 as described above. Patient 12 may be within a hospital such that intubation tube 14 and other materials (inlet filter 171, exhalation valve 171′ and output filter 175) may be provided by the hospital at the time of intubation of patient 12. An optional humidifier unit 302 may be included downstream of respirator module 300a.

Oxygen is delivered to patient 12 via oxygen source 20 by way of electronically actuatable proportional solenoid metering valve 52 before delivery to air/oxygen interface 78′. Inlet pressure regulating valve 304 modulates positive end expiration pressure (PEEP) and may be manually adjustable to suit patient needs. Outlet pressure relief valve 306 regulates/limits the peak inspiratory pressure (PIP) to a doctor-selected patient-dependent value. An additional pressure relief valve 308 may be included for additional safety.

FIG. 5 is an expanded, detailed view of the air/oxygen interface 78′ used within PAP systems 70′ and 170, 300 shown in FIGS. 4, 4D and 5, respectively. Ain 60 represents air stream 24 passing through check valve 54 while Anoz 62 represents pressurized oxygen gas supplied via oxygen source 20 and passing through switching valve 52. The combined gases Ain/Anoz then enter a constricted flow path Amix 64 whereby turbulence and a localized pressure gradient is introduced to ensure thorough mixing of the two gas streams. The combined gas (conditioned air stream 28) then encounters an expanded portion Aout 66, wherein the diameter of Ain 60 is selected to equal the diameter of Aout 66 such that the localized pressure gradient created within Amix 64 is reduced to substantially the same flow rate as air stream 24 entering Ain 60. By way of example and without limitation thereto, Ain 60 may have a diameter of about 0.9 in, Anoz 62 may have a diameter of about 0.025 in, Amix 64 may have a diameter of about 0.182 in. and Aout 66 may have a diameter of about 0.9 in.

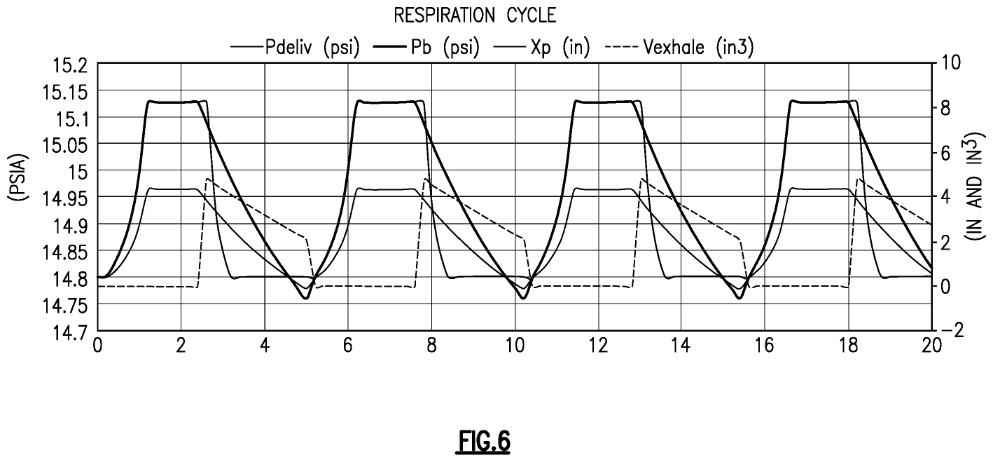

FIG. 6 is a plot of an intubation vent configuration suitable for use with any of PAP systems 70, 70′.

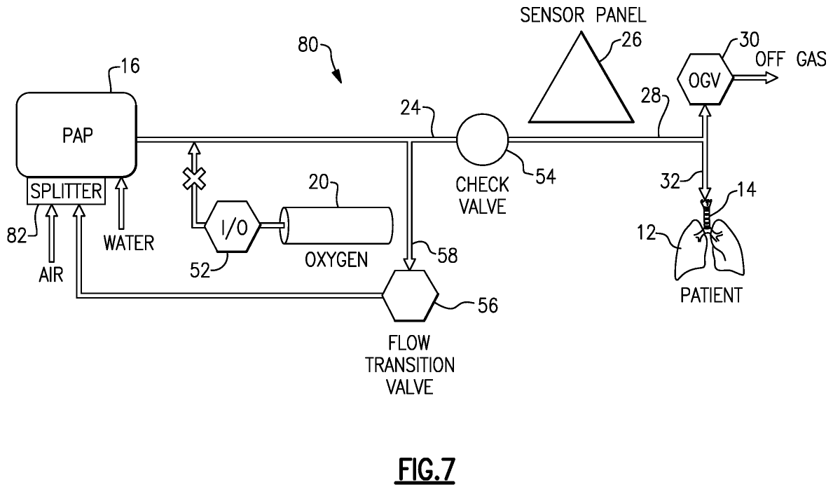

Turning now to FIG. 7, another alternative embodiment of a PAP system is designated generally by the reference numeral 80. PAP system 70 is similar to systems 50 and 70 described above, with the exception of the input location of oxygen from oxygen source 20 and the inclusion of a flow splitter 82 at the inlet to PAP 16. Embodiment 80 places oxygen source 20 (and its associated switching valve 52) downstream of PAP 16 and before check valve 54. In accordance with an aspect of this embodiment, flow transition valve 56 is downstream of oxygen source 20 and upstream of check valve 54. Excess flow 58, which may be upwards of 80%-100%, of air stream 24 may be recirculated to PAP 16 rather than being dumped to ambient. Flow splitter 82 is coupled to the inlet of PAP 16 and can selectively input ambient air and/or recirculated excess flow 58 to PAP 16. As a result, PAP 16 may operate more efficiently with less waste of the supplied oxygen. Downstream of check valve 54 is identical to system 50 (FIG. 2) and operates as described above with respect thereto.

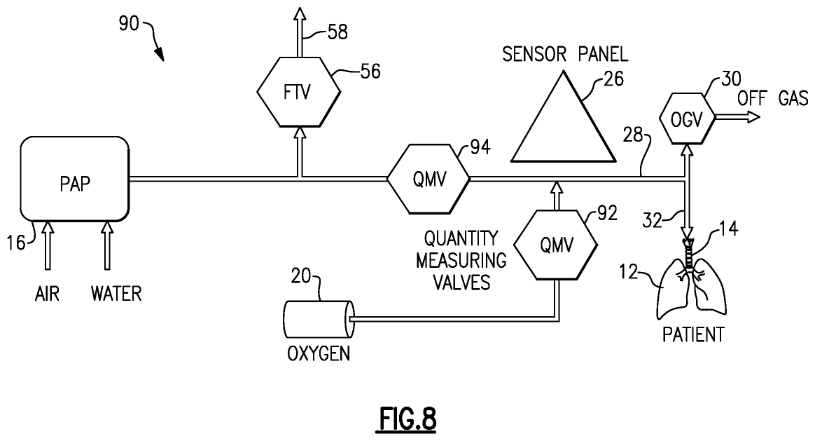

With reference to FIG. 8, another embodiment of a PAP system is designated generally by the reference numeral 90. PAP system 90 is similar to system 70 described above, with switching valve 52 and check valve 54 being replaced with respective quantity measuring valves (QMV) 92 and 94. QMV 94 is placed downstream flow transition valve 56 and upstream of QMV 92 and oxygen source 20. As a result, QMV 94 may input a selected volume of PAP conditioned air, with the excess flow 58 being dumped to ambient via flow transition valve 56. Similarly, a selected volume of oxygen may then be added via QMV 92 before traveling to sensor panel 26.

As described above, sensor unit 26 may be operable to sense one or more characteristics of air stream 24, including but not limited to the pressure, water content (humidity) and/or partial pressure of oxygen within the air stream 24. Should any sensor reading be outside of the set operating parameters of the unit 26, an alarm may sound to alert an attendant of the need for remedial action. The regulated air stream exits unit 26 and is delivered as a conditioned air stream 28 to an off-gas valve 30. Off-gas valve 30 is operable to dump excess gas to ambient via an off-gas port. The resultant conditioned air stream 32 is delivered to the patient 12 via intubation tube 14.

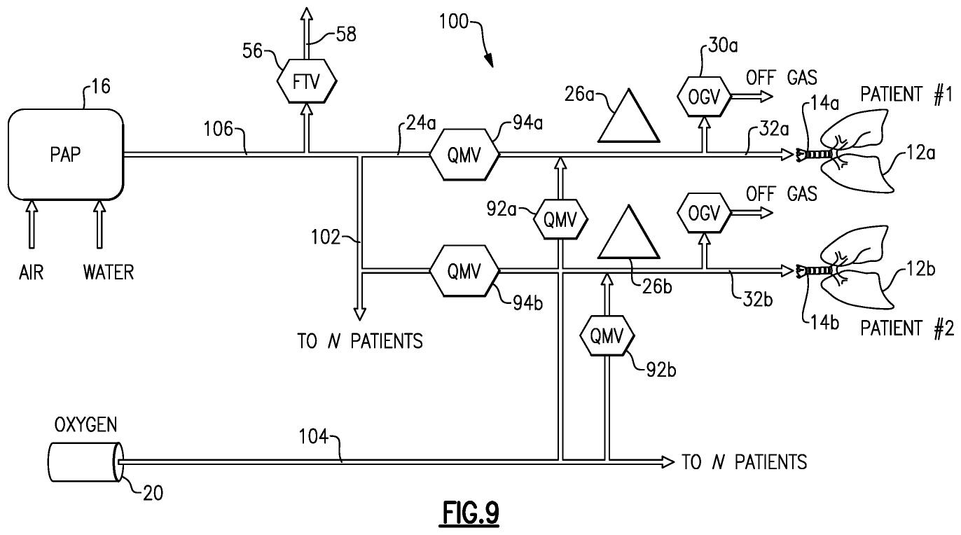

Turning now to FIG. 9, a PAP system is designated generally by the reference numeral 100. PAP system 100 may be configured to assist multiple patients using a single PAP apparatus 16. PAP system 100 is identical to PAP system 90 but includes the further provision of a PAP manifold 102 and oxygen manifold 104. PAP manifold 102 is placed downstream flow transition valve 56 and upstream of QMV 94. PAP manifold is configured to receive PAP conditioned air and divide the flow for delivery to multiple patients 12. Similarly, oxygen manifold 104 is configured to distribute oxygen to multiple patients 12.

With continued reference to FIG. 9, and by way of example and without limitation thereto, system 100 may be set up to supply a dedicated conditioned air stream 32 to two patients 12a and 12b. That is, PAP manifold 102 may split the PAP conditioned air into first and second discrete flows, 24a and 24b, respectively. A volume of each respective flows 24a, 24b may by individually selected via respective QMV 94a, 94b. Similarly, oxygen manifold 104 may split the flow of oxygen from oxygen source 20 with selected volumes being delivered via respective QMV 92a, 92b. In this manner, each patient 12a, 12b may receive a specific air stream 32a, 32b as determined by their doctor or other treating physician.

As describe above, system 100 may also include respective sensor units 26a, 26b and off gas valves 30a, 30b. As a result, each patient receives a dedicated flow of air without cross-contamination. Also, patients may be added or removed online without interrupting supply to any other patients. Further, as indicated, any number of patients may be serviced via system 100. It should be further noted that a plenum 106 may also be included immediately downstream of PAP 16. Plenum 106 may store pressurized PAP conditioned air for distribution to PAP manifold 102 and mitigate demands on PAP production during high-demand occurrences.

From the foregoing, it will be seen that this invention is one well adapted to attain all the ends and objects hereinabove set forth together with other advantages which are obvious and which are inherent to the system and method. It will be understood that certain features and sub combinations are of utility and may be employed without reference to other features and sub combinations. This is contemplated by and is within the scope of the claims. Since many possible embodiments of the invention may be made without departing from the scope thereof, it is also to be understood that all matters herein set forth or shown in the accompanying drawings are to be interpreted as illustrative and not limiting.

The constructions described above and illustrated in the drawings are presented by way of example only and are not intended to limit the concepts and principles of the present invention. As used herein, the terms “having” and/or “including” and other terms of inclusion are terms indicative of inclusion rather than requirement.

While the invention has been described with reference to preferred embodiments, it will be understood by those skilled in the art that various changes may be made and equivalents may be substituted for elements thereof to adapt to particular situations without departing from the scope of the invention. Therefore, it is intended that the invention not be limited to the particular embodiments disclosed as the best mode contemplated for carrying out this invention, but that the invention will include all embodiments falling within the scope and spirit of the appended claims.

What is claimed is:

1) An apparatus configured to supply oxygenated conditioned air to one or more patients, the apparatus comprising:
a) an inlet air source configured to provide an inlet air to the apparatus;
b) a pressurized oxygen source configured to provide a pressurized oxygen gas to the inlet air;
c) a sensor panel including one or more sensors communicatively coupled to each of the inlet air source and the pressurized oxygen source, wherein the sensor panel selectively controls metering of the pressurized oxygen gas from the pressurized oxygen source whereby a partial pressure of the pressurized oxygen gas within the oxygenated conditioned air is maintained within a predetermined concentration range; and
d) an oxygenated conditioned air output configured to couple to a delivery device adapted to supply the oxygenated conditioned air to the one or more patients.

2) The apparatus of claim 1 wherein the oxygenated conditioned air delivery device is a mask, nasal cannula or intubation tube.

3) The apparatus of claim 1 further comprising a mixing device configured to receive each of the inlet air and the pressurized oxygen gas to produce the oxygenated conditioned air.

4) The apparatus of claim 1 wherein the inlet air source is a positive airway pressure (PAP) device selected from a continuous positive airway pressure (CPAP) or bi-level positive airway pressure (BiPAP) or BiPAP Spontaneous Timed (BiPAP ST) device.

5) The apparatus of claim 4 further comprising a mixing device configured to receive each of the inlet air and the pressurized oxygen gas to produce the oxygenated conditioned air.

6) The apparatus of claim 3 wherein the mixing device is included within the PAP device.

7) The apparatus of claim 1 further comprising an off gas valve positioned between the sensor panel and the oxygenated conditioned air delivery device.

8) The apparatus of claim 1 further comprising a check valve positioned before the oxygenated conditioned air delivery device.

9) The apparatus of claim 3 further comprising a flow splitter coupled to the PAP device, wherein the flow splitter is configured to receive ambient air and an excess flow of oxygenated conditioned air to form the inlet air.

10) The apparatus of claim 3 further comprising a first quantity measuring valve (QMV) disposed inline between the PAP device and the sensor panel and configured to output a preselected volume of inlet air to the delivery device, and a second QMV disposed inline between the pressurized oxygen source and the sensor panel and configured to output a preselected volume of pressurized oxygen gas to the oxygenated conditioned air output

11) The apparatus of claim 10 further comprising
a) a PAP manifold including a plurality of inlet air lines, wherein each inlet air line is in fluid communication with a respective oxygenated conditioned air output, and wherein each air line includes a respective first QMV disposed inline between the PAP and a respective sensor panel; and
b) an oxygen manifold including a plurality of pressurized oxygen gas lines, wherein each pressurized oxygen gas line is in fluid communication with a respective air inlet line, and wherein each pressurized oxygen gas line includes a respective second QMV disposed inline between the pressurized oxygen source and each of the respective sensor panels,
wherein oxygenated conditioned air is selectively supplied to one or more of the respective oxygenated conditioned air outputs.

12) The apparatus of claim 1 wherein each of the inlet air source, pressurized oxygen source, sensor panel and delivery device are housed within a respective modular unit.

13) A method for supplying oxygenated conditioned air to one or more patients, the method comprising:
a) providing an inlet air source to input inlet air;
b) providing a pressurized oxygen source configured to provide a pressurized oxygen gas to the inlet air;
c) providing a sensor panel including one or more sensors communicatively coupled to each of the inlet air source and the pressurized oxygen source;
d) producing an oxygenated conditioned air by mixing the inlet air with the pressurized oxygen gas;
e) providing an oxygenated conditioned air delivery device adapted to supply the oxygenated conditioned air to the one or more patients; and
f) metering, via the sensor panel, the pressurized oxygen gas within the oxygenated conditioned air to maintain a partial pressure of the pressurized oxygen gas within a predetermined concentration range.

14) The method of claim 13 wherein the oxygenated conditioned air delivery device is a mask, nasal cannula or intubation tube.

15) The method of claim 13 further comprising a mixing device configured to receive each of the inlet air and the pressurized oxygen gas to produce the oxygenated conditioned air.

16) The method of claim 13 wherein the inlet air source is a positive airway pressure (PAP) device selected from a continuous positive airway pressure (CPAP) or bi-level positive airway pressure (BiPAP) or BiPAP Spontaneous Timed (BiPAP ST) device.

17) The method of claim 16 further comprising a flow splitter coupled to the PAP device, wherein the flow splitter is configured to receive ambient air and an excess flow of oxygenated conditioned air to form the inlet air.

18) The method of claim 16 further comprising a first quantity measuring valve (QMV) disposed inline between the PAP device and the sensor panel and configured to output a preselected volume of inlet air to the delivery device, and a second QMV disposed inline between the pressurized oxygen source and the sensor panel and configured to output a preselected volume of pressurized oxygen gas to the oxygenated conditioned air output

19) The method of claim 18 further comprising
a) a PAP manifold including a plurality of inlet air lines, wherein each inlet air line is in fluid communication with a respective oxygenated conditioned air output, and wherein each air line includes a respective first QMV disposed inline between the PAP and a respective sensor panel; and
b) an oxygen manifold including a plurality of pressurized oxygen gas lines, wherein each pressurized oxygen gas line is in fluid communication with a respective air inlet line, and wherein each pressurized oxygen gas line includes a respective second QMV disposed inline between the pressurized oxygen source and each of the respective sensor panels,
wherein oxygenated conditioned air is selectively supplied to one or more of the respective oxygenated conditioned air outputs.

20) The method of claim 13 wherein each of the inlet air source, pressurized oxygen source, sensor panel and delivery device are housed within a respective modular unit
